(19) United States
(12) Patent Application Publication
(10) Pub. No.: US 2006/0147533 A1
(43) Pub. Date: Jul. 6, 2006

(54) Antimicrobial biomaterial for blood bags

(76) Inventors: Vijayashree Balasubramanian, Chicago, IL (US); Susan M. Zawaski, Western Springs, IL (US); Victor Hugo Perez-Luna, Naperville, IL (US)

Correspondence Address: Victor PEREZ-LUNA, 1921 Ridgefield Lane, Naperville, IL, 60540, US

(21) Appl. No.: 11/027,000

(22) Filed: Dec. 31, 2004

(51) Int. Cl. A61K 33/38; C09D 5/16; A61K 9/14

(52) U.S. Cl. 424/486; 424/618; 523/122

(57) ABSTRACT

An antimicrobial biomaterial including a polymer substrate and silver particles bound to at least a portion of a surface of the polymer substrate. The antimicrobial biomaterial is useful in various medical devices, particularly blood or blood component storage bags, that contact or contain human blood, blood components, and/or other bodily fluids.

### FIELD OF THE INVENTION

This invention relates generally to an antimicrobial biomaterial and, more particularly, to medical devices, such as blood or blood component bags, formed from or including the antimicrobial biomaterial.

### BACKGROUND OF THE INVENTION

Recently, attention has been focused to reduce and eliminate the risk of virus transmission through the transfusion of blood and blood components. Nucleic acid testing (NAT) and highly sensitive assays have increased the detection of numerous viruses such as HIV and HCV. Reports in the United States place the risk of HIV infection from transfusion as low as between 1 in 1,930,000 and 1 in 3,500,925. However, concerns remain over the safety of stored blood supplies.

Currently, bacterial contamination of blood and platelets for transfusion typically far exceeds viral contaminations. Approximately twelve million red blood cell (RBC) units and nine million platelet units are transfused in the United States each year. An estimated incidence of bacterial contamination of platelet units is as high as 1 in 1,000 to 1 in 2,000, while an estimated incidence of bacterial contamination of RBC units is as high as about 1 in 30,000. These estimated figures lead to an estimated 3,000 to 4,500 cases of sepsis occurring annually due to bacterial contaminated blood components, typically with several hundred of these case being severe and possibly fatal. Bacterial sepsis from contaminated blood products is typically the second leading cause of transfusion-related deaths in the United States.

Pathogen inactivation strategies for use with stored blood products are in development to offset generally insufficient current bacteria detection methods. Various agents, such as Psoralens, riboflavins, FRALEs, Inactine, and dimethylmethylene blue (DMMB), are being or have been considered for use as pathogen inactivators in stored blood supplies. These agents typically inactivate pathogens by entering into the microorganism and modifying the DNA or RNA machinery. The DNA or RNA modification generally triggers a sequence of events which culminate in the inhibition of pathogen protein synthesis. While these agents may still be being investigated, complications associated with these agents have generally halted their progression to clinical trials.

A primary concern with the above agents in the inability of the agent to differentiate between pathogens and other blood cells (e.g., red blood cells, white blood cells, and/or platelet) that are vital to physiological function. Studies demonstrated that many currently investigated agents nonspecifically destroy leukocytes, leading to the possibility that particular agents may be useful in treating stored platelet and plasma, but not whole blood. There is a need for a single agent that is useful for eradicating pathogens in all types of transfusion products. Furthermore, currently proposed treatment techniques generally require multiple, time consuming steps to achieve pathogen inactivation and/or extra labor and equipment. In addition, any toxic and systematic effects of these agents have generally not yet been fully determined.

There is a need for an improved agent that is effective in reducing or eliminating pathogens in stored blood or blood products. There is a need for a single agent that eradicates pathogens in all types of transfusion products. There is a need for an agent that can be more easily incorporated into blood storage bags.

### SUMMARY OF THE INVENTION

A general object of the invention is to provide an improved biomaterial having antimicrobial functions or properties.

A more specific objective of the invention is to overcome one or more of the problems described above.

The general object of the invention can be attained, at least in part, through an antimicrobial biomaterial including a polymer substrate and silver particles bound to at least a portion of a surface of the polymer substrate. The antimicrobial biomaterial is useful in various medical devices that contact or contain human blood, blood components, and/or other bodily fluids.

The prior art generally fails to provide a relatively low cost, safe, and efficient biomaterial having antimicrobial properties.

The invention further comprehends a blood or blood component storage bag comprising a biomaterial. The biomaterial includes silver particles bound to at least a portion of a surface of the biomaterial.

The invention still further comprehends a blood or blood component storage bag comprising a polymer substrate, diamines bonded to at least a portion of a surface of the polymer substrate, and a silver particle attached to an amine group of each of at least a plurality of the diamines.

As used herein, references to a “silver particle” are to be understood to refer to a particle containing two or more silver ions.

Further, references herein to “colloidal silver” are to be understood to refer to particles of silver having a dimension of 1-1,000 nanometers.

References herein to “bound” or “bind” are to be understood to refer to an indirect or direct combination of two materials or compounds, desirably by chemical action.

References herein to “bond” or “bonded” are not intended to be limited to any one particular type of chemical bonding, such as ionic chemical bonding or covalent chemical bonding.

References herein to “attach” or “attached” are to be understood to refer to a direct connection of two materials or compounds by either ionic chemical bonding, covalent chemical bonding, differences in surface or other charges, e.g., electrostatic interaction, or other chemical action.

Other objects and advantages will be apparent to those skilled in the art from the following detailed description taken in conjunction with the appended claims and drawings.

### BRIEF DESCRIPTION OF THE DRAWINGS

FIG. 1 is a plan view of a representative blood or blood component bag according to one embodiment of this invention.

### DETAILED DESCRIPTION OF THE INVENTION

The present invention provides an antimicrobial biomaterial that is useful in medical devices, and more particularly medical devices that store, transport, and/or contact blood, blood components, and/or other bodily fluids. The antimicrobial biomaterial is bound with silver particles, such as colloidal silver, which are effective antimicrobial agents. The antimicrobial biomaterial can be obtained by forming a biomaterial, such as is already known in the art, and binding silver particles, as discussed further below, to at least a portion of one or more surfaces of the biomaterial. The biomaterial can then be used to produce medical devices, such as blood and blood component storage bags, surgical tubing, catheters, and cardiovascular devices such as grafts, stents, and sternum plates, in accordance with, for example, known and useful techniques already available in the art for known biomaterials.

In one embodiment of this invention, the antimicrobial biomaterial includes a polymer substrate and silver particles bound to at least a portion of one or more surfaces of the polymer substrate. In one particularly preferred embodiment of this invention, the silver particles comprise colloidal silver. In one embodiment of this invention, the silver particles desirably have an average particle radius of about 1.0 to about 10 nanometers, and more desirably about 5 nanometers.

The polymer substrate of this invention can be formed of any polymer known and/or used in the art for forming medical devices for storing, holding, contacting, and/or transporting blood, blood components, and/or other bodily fluids. In one preferred embodiment of this invention, the polymer substrate includes, for example, polymers such as polyvinyl chloride, poly(ethylene-co-vinyl acetate), fluorinated polyethylene propylene, or combinations thereof. As will be appreciated by one skilled in the art following the teachings herein provided, various and alternative polymers are available as the polymer substrate of this invention and can be bound with silver particles according to this invention.

In one embodiment of this invention, the silver particles are bound to the polymer substrate surface by silver attachment compounds. The silver attachment compounds are chemical compounds to which the silver particles attach, thereby binding the silver particles to the polymer substrate. The silver attachment compounds bond, such as by ionic chemical bonding, to the polymer substrate surface, and the silver particles attach to the silver attachment compounds. The silver attachment compounds desirably each include at least one silver attachment group, wherein the silver particles attach to the silver attachment compounds through the silver attachment groups. Silver particles, such as colloidal silver, prepared by wet chemical synthesis, such as, for example, through reduction of soluble silver salts, generally possess a net negative charge. The negative charge is or can be imparted by the co-adsorption of anions present in the aqueous system. Therefore, silver attachment groups with a positive charge can facilitate the attachment of silver particles through, for example, electrostatic interactions. Thus, the silver attachment groups desirably include as a silver attachment group a chemical or functional group that provides an opposing positive charge to allow for attachment due to the difference in charge.

Examples of silver attachment groups, i.e., chemical or functional groups that the silver particles attach to, include, without limitation, an amine group, a sulfide group, a thiol group, and combinations thereof. As will be appreciated by those skilled in the art following the teachings herein provided, other chemical or functional groups that attach silver particles, and compounds including such groups, e.g., sulfides, disulfides, and thiols, are available for use in this invention. In one embodiment of this invention, amine groups are preferred as the silver attachment compound. Amine groups are or can be advantageous due to the positive charge exhibited at neutral pH. In addition, the nitrogen of the amine group possesses unpaired electrons that allow the formation of a coordinated bond with silver, thereby promoting attachment of silver particles and providing an attachment that is generally more stable than that achieved by only electrostatic interactions. Thiol groups are also known to have a strong affinity for silver compounds, and can establish strong attachment with the silver particles of this invention.

The silver attachment compound of this invention desirably includes another chemical or functional group that can be identical to, similar to, or different from the silver attachment group. This other or second chemical or functional group provides for bonding of the silver attachment compound to the polymer substrate surface. Particular polymer substrates for use in this invention are nonreactive with the silver attachment compounds. The polymer substrate surface can be altered or treated to provide the ability to react with the silver attachment compounds to bond the silver attachment compounds to the surface.

In one embodiment of this invention, the polymer substrate surface is oxidized to allow or promote bonding with the silver attachment compounds. The polymeric substrate surface can be oxidized by any means known and available in the art. In particularly preferred embodiments of this invention, the polymeric substrate surface is oxidized by plasma treatment (e.g., a plasma etching process using oxygen), sulfuric acid treatment, and/or hydrogen peroxide treatment of at least a portion of the polymeric substrate surface. Oxidizing the polymer substrate surface introduces oxygen containing chemical groups, such as carboxyl groups. Further examples of surface oxidation methods include radio frequency glow discharge plasma etching with air/oxygen, corona treatment (an electric discharge created in the vicinity of the polymeric substrate surface), and/or treatment with chromic acid or piranha solution (7/3 concentrated H₂SO₄ and 30% H₂O₂). These procedures generally create highly reactive materials or species and oxygen radicals that combine with the carbon present in the backbone of the polymers, thereby producing a variety of carbon/oxygen compounds (e.g., hydroxyl, carboxyl, ketone, peroxides, etc.). Among the carbon/oxygen compounds, at least the carboxyl groups serve as reactive or functional groups for the attachment of amines through formation of amine bonds, such as by using peptide coupling reagents, such as, for example, carbodiimides and N-hydroxysuccinimide.

Thus the oxidizing treatment of the polymeric substrate surface desirably results in chemical or functional groups on the substrate surface to which a corresponding chemical or functional group of the silver attachment compounds can bond. The silver particles can desirably be attached to the polymeric substrate surface by dipping or immersing, spraying, or otherwise contacting the treated or oxidized portion of the surface in or with aqueous colloidal silver, such as, for example, aqueous colloidal silver having colloidal silver concentrations in the nanomolar or picomolar range.

As discussed above, in one embodiment of this invention, the oxidizing treatment of the polymeric substrate surface, such as polyvinyl chloride, produces or results in the formation of a plurality of carboxyl groups. Each of the silver attachment compounds includes a chemical or functional group that bonds to one of the carboxyl groups. In one preferred embodiment of this invention, the silver attachment compounds are diamines. The diamines each include two amine groups. One amine group bonds with the carboxyl group, thereby bonding the diamine to the polymeric substrate surface. The other amine group is or functions as a silver attachment group to which a silver particle attaches. The silver particle is thus bound to the polymeric substrate by or through the diamine silver attachment compound.

In one embodiment of this invention, the amine functionalities, e.g., diamine silver attachment compounds, are introduced to the oxidized polymer substrate surface by first activating the carboxyl groups on the oxidized polymer substrate surface. The carboxyl groups may be activated by treatment with an aqueous solution of 1-ethyl-3-(3-dimethylaminopropyl)carbodiimide (e.g., 200 mM) and N-hydroxysuccinimide (e.g., 50 mM), such as for a time frame of about, for example, 15 minutes. The surface is then rinsed with distilled water and treated with diamines, such as, for example, 2,2′-(ethylenedioxy)bis(ethylene-diamine) (e.g., 1 mM in water), such as for a time frame of, for example, about one hour. As discussed above, amine groups of the diamine molecules react with the carboxyl groups to bond the diamine molecule to the surface. As will be appreciated, other peptide coupling reagents are also available for use in bonding diamines to the oxidized surface. Examples of other suitable reagents for use in bonding diamines to the oxidized surface include pentafluorophenol (instead of N-hydroxysuccinimide) or Woodward's reagent K (instead of carbodiimide). As will be appreciated by those skilled in the art following the teachings herein provided, various and alternative means or methods can be used for binding the silver particles to the polymeric substrate. For example, other chemical groups on the oxidized polymer substrate surface can be used to bond amine molecules. For example, surface hydroxyl groups can be activated with epichlorohydrin to accept epoxy groups on the surface, which can further react with diamine molecules. Similarly, other silver attachment compounds such as thiols or disulfides may be introduced by using heterofunctional molecules that posses one chemical group that reacts with the oxidized polymer substrate surface and another chemical group that could be a thiol. In addition, in one embodiment of this invention, the silver particles are bound directly to the oxidized surface, such as by bonding to the carboxyl groups on the oxidized surface.

The silver particles bound to the polymeric substrate provide an effective and beneficial antimicrobial agent, thereby providing an antimicrobial biomaterial. Silver particles, such as colloidal silver, provide a mechanism that destroys prokaryotic organisms, such as bacteria, while not affecting eukaryotic cells, such as red blood cells and leukocytes. The silver particles thus provide an advantage over other antimicrobial agents that indiscriminately destroy prokaryotic cells and eukaryotic cells. Silver ions released from the silver particles, such as upon contact with blood, blood components, or other aqueous fluids, inactivate the Na⁺/K⁺ ATPase enzyme on many, and desirably substantially all, cells in the blood, blood components, or other contacting fluids. However, eukaryotic cells alone generally have the necessary cellular mechanism to reverse this inactivation. Eukaryotic cells possess free cysteine or cysteine analogs on or in their cell membranes that readily bind to the silver, thereby reactivating the Na⁺/K⁺ ATPase enzyme on the cell membranes and preventing any damage. Prokaryotic cells generally do not include any free cysteine or cysteine analogs associated with the cell membranes. Therefore, the silver can rapidly reduce or eliminate pathogens, particularly from blood or blood components in contact with the antimicrobial biomaterial.

Other advantages or benefits of the antimicrobial biomaterial of this invention are also apparent. One advantage of the antimicrobial biomaterial of this invention is that it can be sterilized without affecting the antimicrobial properties of the biomaterial. Whereas other antimicrobial agents may be destroyed or altered upon a necessary sterilization before shipment or use, the silver particles are generally not affected by sterilization. Sterilization by high heat may actually cause or promote release of the bound silver particles from the polymeric surface and/or silver ions from the silver particles. The release of silver can beneficially accelerate contact between the silver and more of the blood or blood components. Another advantage is that the processes for making medical devices, such as blood and blood component bags, from the antimicrobial biomaterial of this invention would generally remain the same as those used with currently available biomaterials. In one embodiment of this invention, the only additional step(s) beyond known manufacturing process in forming a medical device is the treatment of the biomaterial to bind the silver particles.

The silver particles are effective in relatively low levels, thereby reducing or eliminating risk of toxicity. Silver has been shown to be effective at killing bacteria in concentrations as low as between about 1 and 10 micromolar. Silver is known to be tolerated by the human body, and silver has been used for many years as a treatment for particular pathological conditions. The estimated fatal dose of silver nitrate in humans is 10 grams, but recoveries have been reported following ingestion of higher doses. A concentration of colloidal silver of about 10 to about 25 parts per million (i.e., one milligram of silver particles per liter of solution) will generally effectively reduce or eliminate bacteria and not be toxic to eukaryotic cells. In one embodiment of this invention, no more than a maximum of 7 milligrams of silver is used for a 250 milliliter blood bag. Methods of applying the silver particles to the biomaterial surface according to this invention typically have an inherent upper limit to the number of silver particles that can be attached, thereby generally providing an inherent safety threshold. In one embodiment of this invention, using an average silver particle size of about 5 nanometers (radius), and taking into account that random sequential adsorption theory predicts a maximum coverage of 55%, the amount of bound silver is about 5×10⁻⁷ g/cm². The relatively low doses (micromolar ranges) of silver from, and/or the short-term use of, the medical devices of this invention generally should not raise concern of toxicity or conditions related to high levels of silver contact, such as argyria.

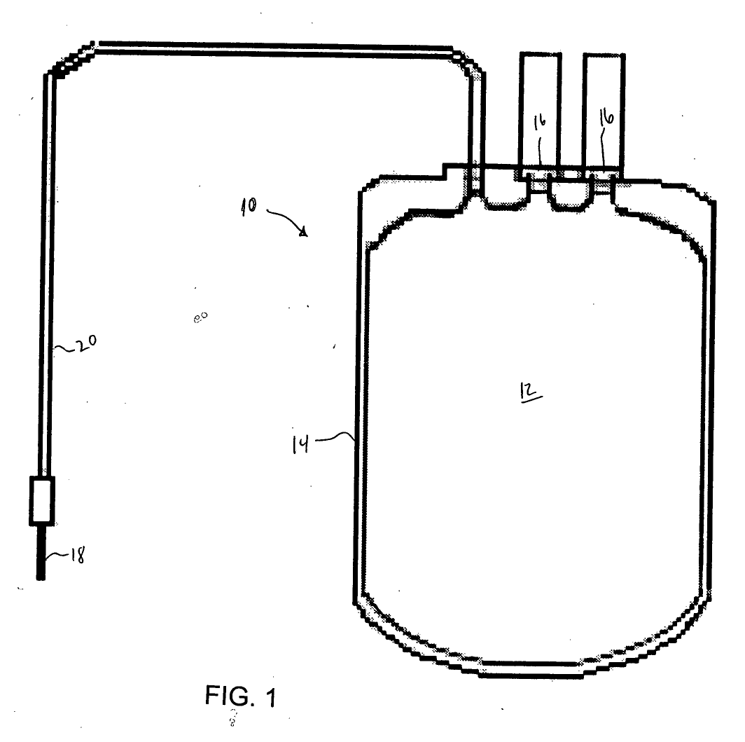

As discussed above, the antimicrobial biomaterial is useful in devices that contact or contain bodily fluids, and where infection is possible. The antimicrobial biomaterial of this invention is particularly useful in medical devices that contain polymeric materials that contact or contain blood or blood components. An example of a medical device formed of the antimicrobial biomaterial of this invention is shown in FIG. 1. FIG. 1 is a general representation of a blood or blood component storage bag, referred to hereinafter as a “blood bag,” known and available in the art. As will be appreciated by those skilled in the art, this invention is not intended to be limited to any particular configuration of blood bag, as the antimicrobial material of this invention can be incorporated into any known blood bag or other medical device formed of or including a polymeric biomaterial.

The blood bag 10 encloses a chamber 12 for containing or storing blood or blood components. The blood bag 10 is sealed around a peripheral edge 14 to enclose the chamber 12. The chamber 12 is accessible by a pair of sealable openings 16 for filling the chamber 12 with blood, blood components, and/or other materials. A needle 18 is attached to the blood bag 10 by tubing 20 to transport the contents of chamber 12 to a patient. At least a portion of an inner surface of the blood bag 10 includes silver particles bound thereon. As will be appreciated, the inner surface is the surface facing the chamber 12 and it is a surface of the biomaterial. As only the inner surface contacts the blood or blood components contained within the chamber 12, only the inner surface includes bound silver particles. The tubing 20 can also be formed of an antimicrobial biomaterial of this invention. The inner surface of the polymeric tubing can include bound silver particles that reduce or eliminate pathogens during intravenous introduction of the blood or blood components from the blood bag 10 to a patient through the needle 18.

The silver particles can be bound to the biomaterial of the blood bag either before or after assembling the biomaterial into the blood bag 10. In one embodiment of this invention, the blood bag 10 is formed of polyvinyl chloride, although other polymeric substrate materials known in the art for use in forming such blood bags can be used, such as, for example, poly(ethylene-co-vinyl acetate) (EVA). Upon an oxidizing treatment, such as discussed above, a plurality of diamines are bonded to at least a portion of a surface of the polyvinyl chloride substrate. One of the amine groups bonds to carboxyl groups formed by the oxidizing treatment. A plurality of silver particles, desirably introduced to the surface as aqueous colloidal silver, attach to amine groups, and desirably and more particularly, one silver particle per one amine group. An overall negative surface charge of the silver particles causes the silver particle to attach to the positively charged amine group. The silver particles can be introduced to the diamines through colloidal silver, by means such as, for example, dipping the biomaterial in aqueous colloidal silver, spraying the surface with aqueous colloidal silver, or filling and emptying the chamber 12 with aqueous colloidal silver. When, for example, blood is introduced into the chamber 12, silver ions release from the silver particles into the blood. The silver inactivates the Na⁺/K⁺ ATPase enzyme on pathogen cell membranes, thereby reducing or eliminating infectious pathogens in the blood.

Thus, the invention provides an effective antimicrobial biomaterial for use in medical devices that contact blood, such as blood storage bags. The silver particles bound to the biomaterial reduce or eliminate prokaryotic pathogens while having little or no effect on eukaryotic cells, such as blood cells. The antimicrobial functions of the silver particles are generally unaffected by sterilization, and may actually be enhanced by high heat sterilization.

The invention illustratively disclosed herein suitably may be practiced in the absence of any element, part, step, component, or ingredient which is not specifically disclosed herein.

While in the foregoing detailed description this invention has been described in relation to certain preferred embodiments thereof, and many details have been set forth for purposes of illustration, it will be apparent to those skilled in the art that the invention is susceptible to additional embodiments and that certain of the details described herein can be varied considerably without departing from the basic principles of the invention.

What is claimed is:

1. An antimicrobial biomaterial, comprising:
a polymer substrate; and
silver particles bound to at least a portion of a surface of the polymer substrate.

2. The antimicrobial biomaterial according to claim 1, wherein the polymer substrate comprises polyvinyl chloride, poly(ethylene-co-vinyl acetate), fluorinated polyethylene propylene, or combinations thereof.

3. The antimicrobial biomaterial according to claim 1, further comprising silver attachment compounds bonded to the polymer substrate, wherein the silver particles attach to at least a plurality of the silver attachment compounds to bind to the polymer substrate.

4. The antimicrobial biomaterial according to claim 3, wherein the silver attachment compounds each comprise at least one silver attachment group, wherein the silver particles attach to the silver attachment compounds through the silver attachment groups.

5. The antimicrobial biomaterial according to claim 4, wherein the at least one silver attachment group is selected from a group consisting of an amine group, a sulfide group, a thiol, and combinations thereof.

6. The antimicrobial biomaterial according to claim 3, wherein the silver attachment compounds are bonded to the polymer substrate surface upon one of plasma treatment, sulfuric acid treatment, and hydrogen peroxide treatment of the at least a portion of the surface.

7. The antimicrobial biomaterial according to claim 1, further comprising a plurality of silver attachment compounds each including at least one amine group and bonded to the at least a portion of the surface of the polymer substrate, wherein the silver particles attach to at least a plurality of the amine groups to bind the silver particles to the at least a portion of the surface of the polymer substrate.

8. The antimicrobial biomaterial according to claim 7, wherein the plurality of silver attachment compounds comprise diamines bonded to carboxyl groups on the at least a portion of the surface of the polymer substrate.

9. The antimicrobial biomaterial according to claim 1, wherein the silver particles are bound to the at least a portion of the surface of the polymer substrate by a method comprising:
forming carboxyl groups on the at least a portion of the surface of the polymer substrate;
bonding a diamine to each of at least a plurality of the carboxyl groups; and
attaching a silver particle to an amine group of each of at least a plurality of the diamines to bind the silver particles to the at least a portion of the surface of the polymer substrate.

10. The antimicrobial biomaterial according to claim 9, further comprising oxidizing the at least a portion of the surface of the polymer substrate to form the carboxyl groups.

11. The antimicrobial biomaterial according to claim 10, wherein oxidizing the at least a portion of the surface of the polymer substrate comprises plasma treatment, sulfuric acid treatment, or hydrogen peroxide treatment.

12. A medical device comprising the antimicrobial biomaterial of claim 1.

13. A blood or blood component storage bag comprising a biomaterial including silver particles bound to at least a portion of a surface of the biomaterial.

14. The blood or blood component storage bag according to claim 13, wherein the biomaterial comprises polyvinyl chloride, poly(ethylene-co-vinyl acetate), fluorinated polyethylene propylene, or combinations thereof.

15. The blood or blood component storage bag according to claim 13, further comprising a plurality of silver attachment compounds bonded to the surface, the silver attachment compounds each including at least one silver attachment group selected from a group consisting of an amine group, a sulfide group, a thiol, and combinations thereof, wherein the silver particles attach to at least a plurality of the silver attachment groups.

16. The blood or blood component storage bag according to claim 15, wherein the plurality of silver attachment compounds are bonded to the surface upon one of plasma treatment, sulfuric acid treatment, and hydrogen peroxide treatment of the at least a portion of the surface.

17. The blood or blood component storage bag according to claim 13, further comprising diamines bonded to the at least a portion of the surface, wherein the silver particles attach to an amine group of each of at least a plurality of the diamines to bind to the at least the portion of the surface.

18. A blood or blood component storage bag, comprising:
a polymer substrate;
diamines bonded to at least a portion of a surface of the polymer substrate; and
a silver particle attached to an amine group of each of at least a plurality of the diamines.

19. The blood or blood component storage bag according to claim 17, wherein the polymer substrate comprises polyvinyl chloride, poly(ethylene-co-vinyl acetate), fluorinated polyethylene propylene, or combinations thereof.

20. The blood or blood component storage bag according to claim 19, wherein the at least a portion of the surface of the polymer substrate comprises a plurality of carboxyl groups and the diamines are bonded to the carboxyl groups.

21. The blood or blood component storage bag according to claim 20, wherein the at least a portion of the surface of the polymer substrate is oxidized to form the plurality of carboxyl groups.
